(19) United States
(12) Patent Application Publication
(10) Pub. No.: US 2018/0348155 A1
(43) Pub. Date: Dec. 6, 2018

(54) GAS DETECTION APPARATUS

(71) Applicants: NGK SPARK PLUG CO., LTD., Nagoya-shi (JP); Spirosure, Inc., Pleasanton, CA (US)

(72) Inventors: Kazuto MORITA, Iwakura-shi (JP); Hiroyuki NISHIYAMA, Konan-shi (JP); Takahiro YOKOYAMA, Komaki-shi (JP); Masahiro TAKAKURA, Komaki-shi (JP); Tatsunori ITO, Inazawa-shi (JP); Takahisa USHIDA, Nagoya-shi (JP); Solomon SSENYANGE, Fremont, CA (US); Brian AWABDY, Dublin, CA (US); Ryan Richard LEARD, Oakland, CA (US)

(73) Assignees: NGK SPARK PLUG CO., LTD., Nagoya-shi (JP); Spirosure, Inc., Pleasanton, CA (US)

(21) Appl. No.: 15/611,979

(22) Filed: Jun. 2, 2017

(51) Int. Cl. G01N 27/12; G01N 33/00

(52) U.S. Cl. CPC G01N 27/125; G01N 33/0013; G01N 33/0037

(57) ABSTRACT

A gas detection apparatus for detecting the concentration of a first gas component contained in a gas under measurement includes a gas conversion section for convening the first gas component to a second gas component, a gas detection section whose electrical characteristics change with a change in the concentration of the second gas component when the gas detection section is in an activated state, and a detection state setting section. During a detection period, the detection state setting section sets the state of gas detection by the gas detection section to a detection executed state in which the gas detection section can detect the second gas component. During periods which are not the detection period, the detection state setting section sets the state of gas detection by the gas detection section to a detection suspended state in which the gas detection section does not detect the second gas component.

### BACKGROUND OF THE INVENTION

### 1. Field of the Invention

The present invention relates to a gas detection apparatus.

### 2. Description of the Related Art

Gas detection apparatuses for detecting the concentration of a first gas component contained in a gas under measurement are known (see, for example, Japanese Patent Application Laid-Open (kokai) No. H10-300702).

Some such gas detection apparatuses include a gas conversion section and a gas detection section. The gas conversion section converts at least a portion of the first gas component contained in the gas under measurement to a second gas component such that the ratio between the partial pressures of the first gas component and the second gas component coincides with that in the equilibrium state. A converted gas produced as a result of conversion at the gas conversion section is supplied to the gas detection section, and the gas detection section is brought into an activated state in which the gas detection section can detect the second gas component. Thus, the electrical characteristics of the gas detection section change with the concentration of the second gas component in the converted gas.

Such a gas detection apparatus can detect the concentration of the first gas component of the gas under measurement by calculating the concentration of the first gas component based on the concentration of the second gas component of the convened gas detected by the gas detection section.

An example of such a gas detection apparatus is a gas detection apparatus which detects the concentration of NO (a first gas component) contained in a gas under measurement by converting NO to NO₂ (a second gas component).

However, the above-described conventional gas detection apparatus has a problem in that the accuracy in detecting the concentration of the first gas component may be lowered as a result of detecting the first gas component over a long period of time.

In particular, there is a possibility that the gas detection section deteriorates in a certain period after startup of the gas detection apparatus. Specifically, a general practice is that after the startup of the gas detection apparatus, in a certain period of time before gas detection becomes possible, a gas under measurement such as exhaled air is not supplied to the gas sensor and detection of the first gas component is not carried out. However, in such a period, the gas detection section may deteriorate. Namely, even in the period between the startup of the gas detection apparatus and the start of detection of the first gas component, a gas which is not the gas under measurement (e.g., the atmosphere or the like) is supplied to the gas conversion section. As a result, a converted gas which is produced as a result of conversion at the conversion section and from which miscellaneous gases have been removed is supplied to the gas detection section, and a reaction between the converted gas and the gas detection section occurs. Therefore, in a stage before the start of detection of the first gas component, the reaction between the second gas component and the gas detection section occurs, and a particular component (e.g., oxygen ions) accumulates in the gas detection section. As a result, deterioration of the gas detection section is accelerated, and when the deterioration progresses, its accuracy in detecting the first gas component may decrease.

### SUMMARY OF THE INVENTION

It is therefore an object of the present invention to provide a gas detection apparatus which detects the concentration of a first gas component contained in a gas under measurement and which can suppress a decrease in the accuracy in detecting the first gas component over a long period of time.

The above object of the invention has been achieved by providing (1) a gas detection apparatus which is adapted to detect the concentration of a first gas component contained in a gas under measurement and which comprises a gas conversion section, a gas detection section, and a detection state setting section.

The gas conversion section is configured to convert at least a portion of the first gas component contained in the gas under measurement to a second gas component such that the ratio between partial pressures of the first gas component and the second gas component coincides with that in the equilibrium state. The gas detection section, to which a converted gas produced as a result of conversion at the gas conversion section is supplied, is configured such that the electrical characteristics of the gas detection section change with a change in the concentration of the second gas component in the converted gas when the gas detection section is brought into an activated state in which the gas detection section can detect the second gas component. The detection state setting section is configured to set the state of gas detection by the gas detection section.

During a detection period in which the first gas component is detected, the detection state setting section sets the state of gas detection by the gas detection section to a detection executed state in which the gas detection section can detect the second gas component of the converted gas. During periods which are not the detection period, the detection state setting section sets the state of gas detection by the gas detection section to a detection suspended state in which the gas detection section cannot detect the second gas component of the converted gas.

As described above, by setting the state of gas detection by the gas detection section to the detection suspended state during periods which are not the detection period, it becomes possible to shorten the period of time during which the state of gas detection by the gas detection section becomes the detection executed state, as compared with the case where the state of gas detection by the gas detection section is set to the detection executed state immediately after startup of the gas detection apparatus. As a result, since the reaction between the converted gas and the gas detection section does not occur in periods which are not the detection period, the progress of deterioration of the gas detection section can be restrained, and a decrease in accuracy in detecting the second gas component by the gas detection section can be suppressed.

Accordingly, the gas detection apparatus (1) can suppress a decrease in the accuracy in detecting the second gas component at the gas detection section, and as a result, can suppress a decrease in accuracy in detecting the first gas component.

Notably, the gas detection apparatus may include a computation section which computes the concentration of the first gas component in the gas under measurement based on the concentration of the second gas component in the converted gas detected by the gas detection section. Thus, the gas detection apparatus can detect the concentration of the first gas component contained in the gas under measurement.

As used herein, the term “detection suspended state” is a general term which encompasses various states in which the reaction between the converted gas and the gas detection section is prevented from occurring. One example of the detection suspended state is a state in which the converted gas is not supplied to the gas detection section irrespective of whether or not the gas detection section can detect the second gas component. Other examples of the detection suspended state include “1) a state in which in place of the converted gas, a gas not to be detected (e.g., the atmosphere) is supplied to the gas detection section so that the converted gas does not come into contact with the gas detection section” and “2) a state in which the gas detection section has been brought into a state in which it cannot detect the second gas component so that even when the converted gas is supplied to the gas detection section, the reaction between the converted gas and the gas detection section does not occur.”

In a preferred embodiment (2), the gas detection apparatus (1) above further comprises a supply state changeover section which switches the state of gas supply to the gas detection section to either of a supply executed state in which the converted gas is supplied to the gas detection section and a supply suspended state in which the convened gas is not supplied to the gas detection section, and a gas not to be detected (which is not the converted gas) is supplied to the gas detection section. During the detection period, the detection state setting section controls the supply state changeover section such that the state of gas supply to the gas detection section is set to the supply executed state, and during periods which are not the detection period, the detection state setting section controls the supply state changeover section such that the state of gas supply to the gas detection section is set to the supply suspended state.

As described above, as a method of controlling the state of gas detection by the gas detection section to the detection executed state or the detection suspended state, the detection state setting section may employ, for example, a method of switching the state of gas supply to the gas detection section to the supply executed state or the supply suspended state by controlling the supply state changeover section. During periods which are not the detection period, the state of gas supply to the gas detection section is switched to the supply suspended state in which in place of the converted gas, the gas not to be detected is supplied to the gas detection section, whereby the reaction between the gas detection section and the converted gas is prevented from occurring. Also, supply of the gas not to be detected to the gas detection section yields an effect of removing a particular component (a component which causes deterioration) accumulated in the gas detection section. The gas not to be detected may be a gas which removes a deterioration causing substance from the gas detection section. For example, in the case where the gas detection section is configured through use of a sensor element for detecting NOx, the deterioration causing substance may be oxygen ions. In this case, by supplying the ambient atmosphere to the gas detection section as the gas not to be detected, oxygen ions can be removed from the gas detection section, whereby deterioration of the gas detection section can be mitigated or the gas detection section can be recovered from the deteriorated state.

In another preferred embodiment (3) of the gas detection apparatus (2) above, the supply suspended state is a state in which the gas not to be detected is supplied to the gas detection section from the downstream side of the gas detection section, and the gas not to be detected which has passed through the gas detection section is supplied to the gas conversion section.

Such a state is an example of the supply suspended state. Such a supply suspended state can be readily realized by changing the gas moving direction in a gas flow channel between the gas conversion section the gas detection section from the gas moving direction in the supply executed state to the opposite direction (the gas moving direction in the supply suspended state).

In yet another preferred embodiment (4) of the gas detection apparatus (2) above, the supply suspended state is a state in which the supply of the converted gas to the gas detection section is stopped, and the gas not to be detected is supplied to a passage between the gas conversion section and the gas detection section (i.e., the gas not to be detected is supplied to the gas detection section from a position located upstream of the gas detection section and downstream of the gas detection section).

Such a state is an example of the supply suspended state. Such a supply suspended state can be readily realized, for example, by stopping the supply of the converted gas from the gas conversion section to the gas detection section, and supplying the gas not to be detected to the gas flow channel between the gas conversion section and the gas detection section.

In yet another preferred embodiment (5) of the gas detection apparatus (2) above, the supply state changeover section further comprises a conversion state changeover section which switches the state of the gas conversion section to either of a conversion possible state and a no conversion state. The conversion possible state is a state in which the gas supplied to the gas conversion section can be converted to the converted gas. The no conversion state is a state in which the gas supplied to the gas conversion section passes through the conversion section without being converted to the converted gas. During the detection period, the supply state changeover section controls the conversion state changeover section such that the state of the gas conversion section becomes the conversion possible state. During periods which are not the detection period, the supply state changeover section controls the conversion state changeover section such that the state of the gas conversion section becomes the no conversion state.

As described above, as a method of switching the state of gas supply to the gas detection section to the supply executed state or the supply suspended state, the supply state changeover section may employ a method of switching the state of gas conversion by the gas conversion section to the conversion possible state or the no conversion state. Namely, the supply state changeover section can switch the state of gas supply to the gas detection section to the supply executed state or the supply suspended state by switching the state of the gas conversion section to the conversion possible state or the no conversion state by controlling the conversion state changeover section.

Notably, in the case where the gas conversion section is a gas conversion section which becomes the conversion possible state at a conversion possible temperature and becomes the no conversion state at a temperature at which conversion does not take place (i.e., a no conversion temperature), the conversion state changeover section may be configured to switch the temperature of the gas conversion section between the conversion possible temperature and the no conversion temperature. In this case, the supply state changeover section can switch the state of gas supply to the gas detection section to the supply executed state or the supply suspended state by switching the temperature of the gas conversion section to the conversion possible temperature or the no conversion temperature by controlling the conversion state changeover section.

In yet another preferred embodiment (6), the gas detection apparatus of any of (1) to (5) above further comprises a reaction state changeover section which switches the state of the gas detection section to either of a reaction executed state and a reaction suspended state. The reaction executed state is a state in which the gas detection section reacts with the second gas component. The reaction suspended state is a state in which the gas detection section does not react with the second gas component.

The reaction state changeover section sets the state of the gas detection section to the reaction executed state by controlling the temperature of the gas detection section to an activation temperature at which the gas detection section can detect the second gas component and sets the state of the gas detection section to the reaction suspended state by controlling the temperature of the gas detection section to a deactivation temperature at which the gas detection section does not detect the second gas component.

During the detection period, the detection state setting section controls the reaction state changeover section such that the state of the gas detection section becomes the reaction executed state. During periods which are not the detection period, the detection state setting section controls the reaction state changeover section such that the state of the gas detection section becomes the reaction suspended state. During the periods which are not the detection period, the gas detection section is set to a state in which the gas detection section is controlled to the deactivation temperature, whereby the reaction between the gas detection section and the converted gas can be prevented.

In yet another preferred embodiment (7), the gas detection apparatus (6) above further comprises a permission state changeover section which switches the state of gas supply to the gas conversion section between a permission state in which the gas supply is permitted and a prohibition state in which the gas supply is prohibited. The detection state setting section controls the permission state changeover section such that the state of gas supply to the gas conversion section is switched to the permission state during the detection period and is switched to the prohibition state during periods which are not the detection period.

During the periods which are not the detection period, a state in which the gas itself is not supplied to the gas conversion section is established, the converted gas is not produced as a result of passage of the gas through the gas conversion section, whereby the reaction between the gas detection section and the converted gas can be prevented.

In yet another preferred embodiment (8) of the gas detection apparatus of any of (1) to (7) above, the gas conversion section includes a catalyst for replacing NO in the gas under measurement with NO₂ and is configured to convert NO which is the first gas component to NO₂ which is the second gas component, and the gas detection section is configured such that its electrical characteristics change with a change in the concentration of NO₂ which is the second gas component.

One example of the gas detection apparatus is a gas detection apparatus which detects NO as the first gas component and NO₂ as the second gas component.

### BRIEF DESCRIPTION OF THE DRAWINGS

FIG. 1 is a schematic diagram of a gas detection apparatus in the case where the state of its sensor unit is set to a detection executed state;

FIG. 2 is a perspective view of a gas sensor;

FIG. 3 is a cross-sectional view of the gas sensor taken along line of FIG. 2;

FIG. 4 is an exploded perspective view of the gas sensor;

FIG. 5 is a schematic diagram of the gas detection apparatus in the case where the state of its sensor unit is set to a detection suspended state;

FIG. 6 is a schematic diagram of a second gas detection apparatus in the case where the state of its sensor unit is set to a detection executed state;

FIG. 7 is a schematic diagram of the second gas detection apparatus in the case where the state of its sensor unit is set to a detection suspended state;

FIG. 8 is a schematic diagram of a third gas detection apparatus in the case where the state of its sensor unit is set to a detection executed state; and

FIG. 9 is a schematic diagram of the third gas detection apparatus in the case where the state of its sensor unit is set to a detection suspended state.

### DESCRIPTION OF THE PREFERRED EMBODIMENTS

Embodiments to which the present invention is applied will next be described in detail with reference to the drawings. However, the present invention should not be construed as being limited thereto.

### 1. First Embodiment

### 1-1. Overall Structure

A gas detection apparatus 1 for detecting the concentration of NOx (a first gas component) contained in exhaled air (gas under measurement G1) will be described as a first embodiment.

The gas detection apparatus 1 is used to measure NOx contained in exhaled air at a very low concentration (at a level of several ppb to several hundreds of ppb) for the purpose of, for example, diagnosis of asthma.

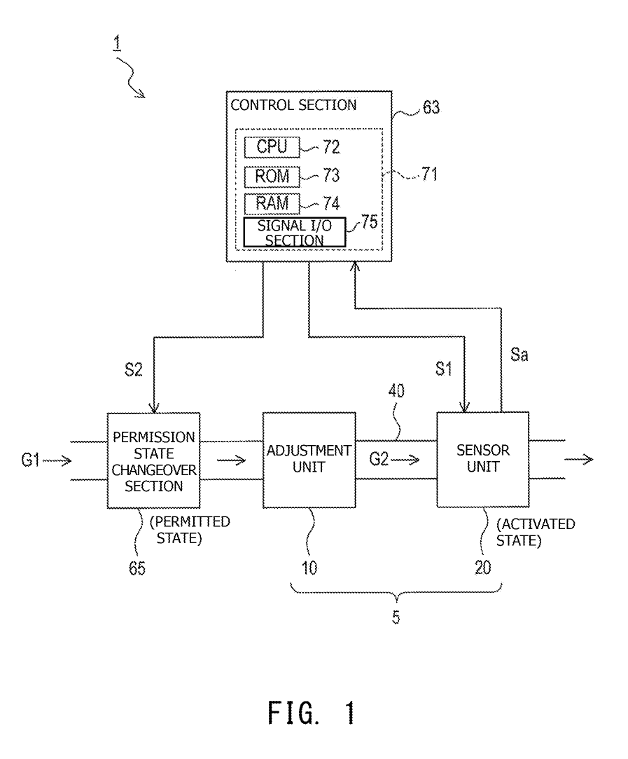

As shown in FIG. 1, the gas detection apparatus 1 includes a gas sensor 5 for measuring NOx contained in the gas under measurement G1, a control section 63 for controlling the gas sensor 5, and a permission state changeover section 65 for switching the state of supply of the gas to the gas sensor 5 (an adjustment unit 10).

The gas sensor 5 includes the adjustment unit 10 and a sensor unit 20.

The adjustment unit 10 includes a catalyst (MCR, Micro Channel Reactor) for converting NO contained in the gas under measurement G1 supplied from the permission state changeover section 65 to NO₂. This catalyst contains, for example, PtY (zeolite which bears Pt) which converts NO to NO₂. The adjustment unit 10 converts at least a portion of NO (the first gas component) contained in the gas under measurement G1 to NO₂ (a second gas component) such that the ratio between the partial pressures of NO and NO₂ coincides with that in the equilibrium state. The adjustment unit 10 supplies to the sensor unit 20 a converted gas G2 which is obtained by adjusting the ratio between the partial pressures of NO and NO₂ in the gas under measurement G1.

The sensor unit 20 includes a mixed-potential sensor element (a sensor element section 24 to be described below) to which the converted gas G2 produced as a result of conversion at the adjustment unit 10 is supplied and which detects NO₂. When the sensor element is brought into an activated state (for example, 400° C.), the sensor element can detect NO₂, and its electrical characteristics change with a change in the detected NO₂ concentration. Namely, the sensor unit 20 is configured such that the converted gas G2 produced as a result of conversion at the adjustment unit 10 is supplied to the sensor unit 20, and its electrical characteristics change with a change in the concentration of NO₂ in the converted gas G2.

The control section 63 is configured to control the state of gas detection by the sensor unit 20 and receive a detection signal Sa which changes with the NO₂ concentration detected by the sensor unit 20 (in other words, the detection signal Sa which changes with the electrical characteristics of the sensor unit 20).

The control section 63 is configured to control at least either of the state of the sensor unit 20 (between an activated state and a deactivated state) and the state of the permission state changeover section 65 when the control section 63 controls the state of gas detection by the sensor unit 20.

The control section 63 can set the state of the sensor unit 20 to the activated state or the deactivated state by controlling the temperature of the sensor unit 20 through output of a first command signal S1. Namely, the control section 63 controls the amount of heat generated by a heater (a first heater 24b to be described below) provided in the sensor unit 20, by controlling the amount of power supplied to the heater through use of the first command signal S1, so as to control the sensor unit 20 (specifically, the sensor element) to an activation temperature (e.g., 400° C. or higher) to thereby set the sensor unit 20 to the activated state. Also, the control section 63 controls the amount of heat generated by the heater, by controlling the amount of power supplied to the heater through use of the first command signal S1, so as to control the sensor unit 20 to a deactivation temperature (e.g., room temperature (25° C. or the like), to thereby set the sensor unit 20 to the deactivated state.

The control section 63 can control the gas supply state of the permission state changeover section 65 by outputting a second command signal S2 so as to switch the state of supply of the gas from the permission state changeover section 65 to the gas sensor 5 (specifically, the adjustment unit 10) to either of a permission state and a prohibited state.

Notably, when set to the permission state, the permission state changeover section 65 opens a gas flow channel provided therein so that the gas can pass through the permission state changeover section 65. When set to the prohibited state, the permission state changeover section 65 closes the gas flow channel provided therein so that the gas cannot pass through the permission state changeover section 65. More specifically, the gas detection apparatus 1 is configured such that when a timing for supplying exhaled air to the gas sensor 5 as the gas under measurement G1 has come after the startup of the gas detection apparatus 1, over a period of time during which detection by the gas sensor 5 is performed, the gas detection apparatus 1 sets the permission state changeover section 65 to the permission state so as to permit the supply of the gas under measurement G1 to the adjustment unit 10. Also, during other periods (namely, periods which are not the detection period), the gas detection apparatus 1 sets the permission state changeover section 65 to the prohibition state so as to prohibit the supply of the gas to the adjustment unit 10.

The permission state of the permission state changeover section 65 is a state in which the gas (the gas under measurement G1) is supplied from the permission state changeover section 65 to the gas sensor 5 (the adjustment unit 10), and is also a state in which the converted gas G2 is supplied from the adjustment unit 10 to the sensor unit 20. The prohibited state of the permission state changeover section 65 is a state in which the gas is not supplied from the permission state changeover section 65 to the gas sensor 5 (the adjustment unit 10), and is also a state in which the converted gas G2 is not supplied from the adjustment unit 10 to the sensor unit 20.

### 1-2. Gas Sensor

Next, the gas sensor 5 will be described.

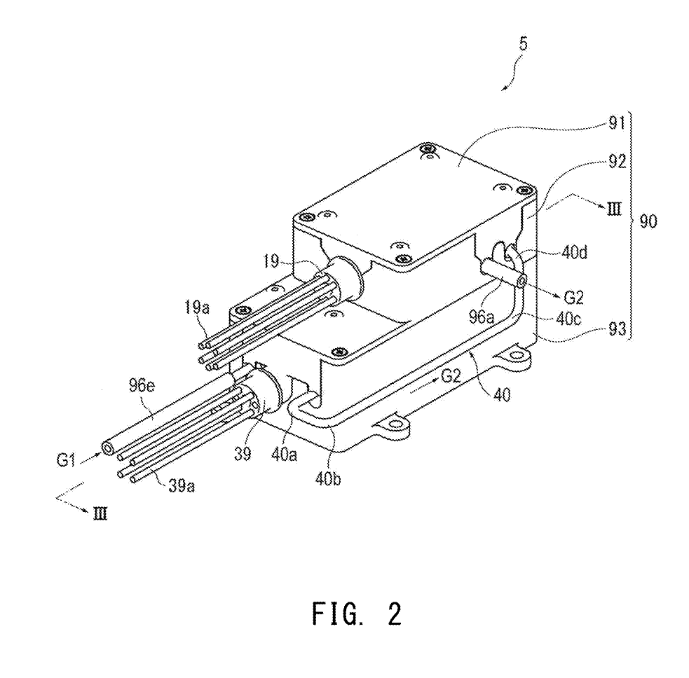

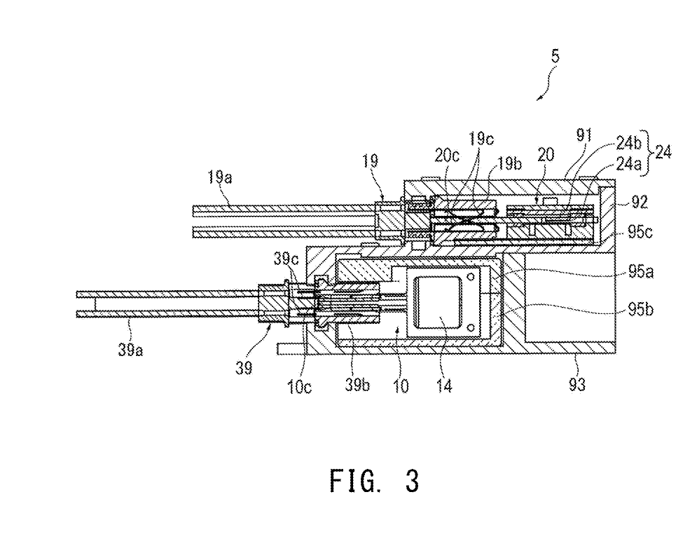

As shown in FIGS. 2 and 3, the gas sensor 5 includes a main body 90 serving as a housing, the adjustment unit 10, the sensor unit 20, and a main pipe 40 (gas flow pipe 40). The adjustment unit 10 and the sensor unit 20 are contained in the main body 90, and the gas sensor 5 has a box-like shape as a whole.

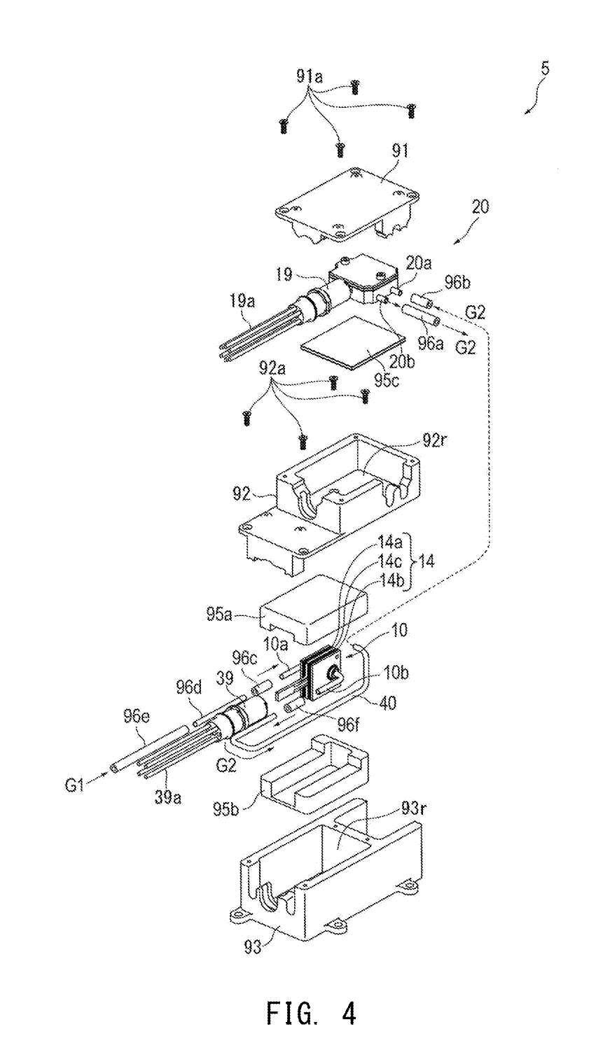

The main body 90 includes a base 93 having an approximately rectangular shape and elongated in the left-right direction in FIG. 2; an upper case 92 having an approximately rectangular shape and shorter in the left-right direction in FIG. 2 than the base 93; and a lid 91 fastened to the upper case 92 with screws 91a to close an internal space 92r of the upper case 92 (see FIG. 4). The main body 90 is formed of a metal or a resin.

One longitudinal end of the upper case 92 (the right end in FIG. 2) is aligned with one longitudinal end of the base 93 (the right end in FIG. 2), and the upper case 92 is fastened to the upper surface of the base 93 with screws 92a to thereby close an internal space 93r of the base 93 (see FIG. 4).

As shown in FIG. 4, the sensor unit 20 is contained in the internal space 92r of the upper case 92, and a tubular cassette connector 19 is connected to the sensor unit 20. The adjustment unit 10 is contained in the internal space 93r of the base 93, and a tubular cassette connector 39 is connected to the adjustment unit 10.

A detection output for a specific component from the sensor unit 20 is taken out to the outside from one end of the cassette connector 19 (the left end in FIG. 2) through lead wires 19a, and heater power for energizing a first heater 24b included in the sensor unit 20 is supplied from the outside through the lead wires 19a. Heater power for energizing a second heater 14c for heating the adjustment unit 10 is supplied to one end of the cassette connector 39 (the left end in FIG. 2) from the outside through lead wires 39a.

As shown in FIG. 2, the gas under measurement G1 is introduced into the adjustment unit 10 inside the base 93 through a sub-pipe 96e, discharged from the adjustment unit 10 and then introduced into the sensor unit 20 inside the upper case 92 by way of the main pipe 40 provided outside the base 93. The sensor unit 20 detects a specific component in the gas under measurement G1, and the gas under measurement G1 is discharged to the outside through a sub-pipe 96a provided outside the upper case 92.

The main pipe 40 protrudes from a front face of the base 93 (the left face in FIG. 2), is bent at a bent portion 40a 90° in the direction of the width of the base 93 (an oblique direction toward the lower right side in FIG. 2), further bent at a bent portion 40b 90° in the lengthwise direction of the base 93 (the direction toward the right side in FIG. 2), and then extends in the lengthwise direction of the base 93. Near the one longitudinal end of the base 93 (the right end in FIG. 2), the main pipe 40 is bent at a bent portion 40c 90° in an upward direction (the upward direction in FIG. 2) toward the upper case 92, bent at a bent portion 40d 90° in the direction of the width of the upper case 92 (an oblique direction toward the upper side in FIG. 2), and then enters the upper case 92.

As described above, the main pipe 40 has at least one bent portion (four bent portions in this example, i.e., the bent portions 40a to 40d). The main pipe 40 is formed from a metal-made pipe (e.g., a stainless steel alloy pipe) having high heat dissipation performance.

Next, the adjustment unit 10 will be described.

The adjustment unit 10 has a box-like shape and contains a conversion section 14. The adjustment unit 10 has an inlet pipe 10a for the gas under measurement G1 which is provided on one side surface thereof, and an outlet pipe 10b for the converted gas G2 which is provided on the other side surface thereof. When the gas under measurement G1 is introduced into the adjustment unit 10 through the inlet pipe 10a, the gas component contained in the gas under measurement G1 is converted to a particular component by the conversion section 14, and the converted gas G2 containing the particular component is discharged to the outside of the adjustment unit 10 through the outlet pipe 10b.

The conversion section 14 is configured to convert the gas component contained in the gas under measurement G1 to the particular component. The conversion section 14 includes a first catalyst 14a, a second catalyst 14b, and the second heater 14c.

The first catalyst 14a and the second catalyst 14b are disposed adjacent to the second heater 14c. The first catalyst 14a and the second catalyst 14b are configured to convert the gas component contained in the gas under measurement G1 to the particular component when they are heated by the second heater 14c. The second heater 14c generates heat upon energization to thereby heat the first catalyst 14a and the second catalyst 14b to a catalyst reaction temperature (a temperature at which the first catalyst 14a and the second catalyst 14b exhibit a catalytic function). The conversion section 14 includes a temperature sensor (not shown) for detecting the heating temperature of the second heater 14c. The temperature sensor has a predetermined pattern.

The first catalyst 14a and the second catalyst 14b can be configured through use of, for example, PtY which convers NO contained in the gas under measurement G1 to NO₂. The second heater 14c can be configured through use of a heat generation element formed in a meandering pattern.

A plurality of conductive pads (not shown) are disposed on the front and back surfaces of a base end portion 10c of the adjustment unit 10. The plurality of conductive pads are electrically connected to the second heater 14c and the temperature sensor (not shown). The second heater 14c generates heat when it is energized by electric power supplied from the outside through the conductive pads.

As shown in FIG. 3, a tubular separator 39b is disposed on the forward end side of the tubular cassette connector 39, and a plurality of spring terminals 39c are held in a plurality of through holes of the tubular separator 39b. When the base end portion 10c of the adjustment unit 10 is inserted into the cassette connector 39, the spring terminals 39c come into elastic contact with the conductive pads of the base end portion 10c and are thereby electrically connected to the conductive pads. Bare forward ends of the lead wires 39a are crimped and fixed to ends of the spring terminals 39c. The rear ends of the lead wires 39a are connected to an unillustrated female connector, and the lead wires 39a are thereby connected to the control section 63.

Namely, in the adjustment unit 10, the gas under measurement G1 comes into contact with the catalyst heated to the catalyst reaction temperature, and the gas component (specifically, NO) contained in the gas under measurement G1 is converted to the particular component (specifically, NO₂), whereby the converted gas G2 is obtained. Specifically, in the adjustment unit 10, the concentrations of NO (the first gas component) and NO₂ (the second gas component) contained in the gas under measurement G1 introduced through the inlet pipe 10a are adjusted (converted) by the conversion section 14, whereby the converted gas G2 is obtained. Namely, after the concentrations of NO and NO₂ are adjusted (converted), the gas under measurement G1 is discharged, as the converted gas G2, to the outside of the adjustment unit 10 through the outlet pipe 10b. The conversion section 14 is a structure which functions to remove miscellaneous gases (e.g., NH₃, H₂, CO, etc.) other than particular gas components (NO (the first gas component) and NO₂ (the second gas component)) and adjust (convert) the concentrations of NO (the first gas component) and NO₂ (the second gas component) in the gas under measurement G1.

Next, the sensor unit 20 will be described.

The sensor unit 20 has a box-like shape and contains a sensor element section 24. The sensor unit 20 has an inlet pipe 20a and an outlet pipe 20b for the converted gas G2 which are provided on the side wall thereof. The converted gas G2 introduced into the sensor unit 20 through the inlet pipe 20a comes into contact with the sensor element section 24, whereby the concentration of the particular component is detected. The converted gas G2 is then discharged to the outside of the sensor unit 20 through the outlet pipe 20b.

The sensor element section 24 includes a detection section 24a and a first heater 24b.

The detection section 24a is configured such that its electrical characteristics change with a change in the concentration of the particular component (NO₂). An electrical signal which changes with a change in the electrical characteristics of the detection section 24a can be used for detecting the concentration of the particular component. The first heater 24b generates heat when energized and heats the detection section 24a to an activation temperature; i.e., operation temperature. Output terminals of the detection section 24a and energization terminals of the first heater 24b are electrically connected to different lead wires 19a. Notably, the detection section 24a includes a temperature sensor (not shown) for detecting the temperature of the first heater 24b. The temperature sensor has a predetermined pattern.

The detection section 24a may be formed as, for example, a mixed potential NOx (nitrogen oxide) sensor including a solid electrolyte layer and a pair of electrodes disposed on surfaces of the solid electrolyte layer. See, for example, US 2015/0250408 incorporated herein by reference in its entirety. The first heater 24b can be configured through use of a heat generation element formed into a meandering pattern. Notably, the detection section 24a may have a known configuration other than the above-described configuration. For example, the detection section 24a may be configured through use of a metal oxide semiconductor.

Conductive pads (not shown) are disposed on a base end portion 20c of the sensor unit 20. The conductive pads are electrically connected to the sensor element section 24 (the detection section 24a and the first heater 24b).

As shown in FIG. 3, a tubular separator 19b is disposed on the forward end side of the tubular cassette connector 19, and a plurality of spring terminals 19c are held in a plurality of through holes of the tubular separator 19b. When the base end portion 20c of the sensor unit 20 where the conductor pads are disposed is inserted into the cassette connector 19, the spring terminals 19c come into elastic contact with the conductive pads and are thereby electrically connected to the conductive pads. Bare forward ends of the lead wires 19a are crimped and fixed to ends of the spring terminals 19c. The rear ends of the lead wires 19a are connected to an unillustrated female connector, and the lead wires 19a are thereby connected to the control section 63.

As shown in FIGS. 3 and 4, the adjustment unit 10 is accommodated in the internal space 93r of the base 93 in a state in which the adjustment unit 10 is covered with an upper heat insulating member 95a from above and with a lower heat insulating member 95b from below. The sensor unit 20 is accommodated in the internal space 92r of the upper case 92 with a sheet-shaped heat insulating member 95c disposed below the sensor unit 20.

Sub-pipes 96c, 96d, and 96e are connected to the inlet pipe 10a of the adjustment unit 10, and one end of the main pipe 40 is connected to the outlet pipe 10b through a sub-pipe 96f. The other end of the main pipe 40 is connected to the introduction pipe 20a of the sensor unit 20 through a sub-pipe 96b, and the sub-pipe 96a is connected to the discharge pipe 20b.

As described above, the adjustment unit 10 and the sensor unit 20 communicate with each other through the main pipe 40 through which the converted gas G2 converted from the gas under measurement G1 can flow. After having flowed into the adjustment unit 10 through the sub-pipe 96e, the gas under measurement G1 flows into the sensor unit 20 through the main pipe 40 and is discharged to the outside through the sub-pipe 96a.

### 1-3. Control Section

The control section 63 includes a microcomputer 71 which executes various types of processes for controlling the gas sensor 5.

The microcomputer 71 includes a CPU 72, a ROM 73, a RAM 74, and a signal input output section 75. The various functions of the control section 63 are realized by a program stored in a non-transitory substantial recording medium and executed by the CPU 72. In this example, the ROM 73 corresponds to the non-transitory substantial recording medium storing the program. Also, as a result of execution of this program, a method corresponding to the program is executed. The signal input output section 75 transmits various signals to the gas sensor 5 (the sensor unit 20), the permission state changeover section 65, external devices (not shown), etc., and receives various signals therefrom. Notably, the number of each of components of the microcomputer 71; i.e., the CPU 72, the ROM 73, the RAM 74, and the signal input output section 75, may be one, two or more. Also, some or all the functions of the microcomputer 71 may be realized by hardware such as one or more ICs or the like.

The control section 63 is configured such that, based on the program stored in the ROM 73, the CPU 72 executes various processes for controlling the gas sensor 5.

For example, as one of the various processes, the control section 63 executes a process of setting the state of the sensor unit 20 to either of the activated state and the deactivated state by controlling the temperature of the sensor unit 20 through output of the first command signal S1 (hereinafter this process will also be referred to as a “sensor state setting process”).

Also, as one of the various processes, the control section 63 executes a process of switching the gas supply state of the permission state changeover section 65 to either of the permission state and the prohibited state by outputting the second command signal S2 (hereinafter this process will also be referred to as a “gas supply changeover process”).

Further, as described above, the control section 63 is configured to receive the detection signal Sa which changes with the detected NO₂ concentration. As one of the various processes, the control section 63 executes a process of computing the NO₂ concentration and the NO concentration in the gas under measurement (exhaled air) based on the detection signal Sa (hereinafter this process will also be referred to as a “concentration computation process”).

In the concentration computation process, the control section 63 computes the NO₂ concentration in the convened gas G2 based on the detection signal Sa and computes the NO concentration in the converted gas G2 based on the computed NO₂ concentration while using the partial pressure ratio between NO and NO₂ adjusted by the adjustment unit 10. As a result, the concentrations of the particular gas components (NO and NO₂) in the converted gas G2 can be obtained, and, based on these concentrations, the concentrations of the particular gas components (NO and NO₂) in the gas under measurement G1 are computed.

Namely, by executing the concentration computation process, the control section 63 can compute the NO concentration in the gas under measurement G1 based on the NO₂ concentration in the converted gas G2 detected by the sensor unit 20.

The control section 63 transmits to an external device regarding the concentrations of the particular gas components (NO and NO₂) obtained as a result of executing the concentration computation process. The control section 63 transmits the information regarding the concentrations of the particular gas components to a display, an information storage device, or the like which serves as an external device. The external device having received the information executes various processes (display, data storage, etc.) through use of the information regarding the concentrations of the particular gas components.

Further, as one of the various processes, the control section 63 executes a process of setting the state of gas detection by the sensor unit 20 depending on whether or not the present period is an NO detection period (hereinafter this process will also be referred to as a “detection state setting process”). In the detection state setting process, when the present period is the NO detection period. the control section 63 sets the state of gas detection by the sensor unit 20 to a detection executed state in which the sensor unit 20 can detect NO₂ contained in the converted gas G2. When the present period is not the NO detection period, the control section 63 sets the state of gas detection by the sensor unit 20 to a detection suspended state in which the sensor unit 20 does not detect NO₂ contained in the converted gas G2.

In the case where the control section 63 determines, during execution of the detection state setting process, that the present period is the NO detection period, for setting the state of gas detection by the sensor unit 20 to the detection executed state, the control section 63 outputs the first command signal S1 so as to set the state of the sensor unit 20 to the activated state and outputs the second command signal S2 so as to set the gas supply state of the permission state changeover section 65 to the permission state (the gas supply changeover process). As a result, as shown in FIG. 1, the gas detection apparatus 1 can set the state of the permission state changeover section 65 to a state in which the gas under measurement G1 can pass through the permission state changeover section 65, whereby it becomes possible to supply to the sensor unit 20 the converted gas G2 converted from the gas under measurement G1 at the adjustment unit 10. Therefore, the gas detection apparatus 1 can detect NO₂ of the converted gas G2 at the sensor unit 20.

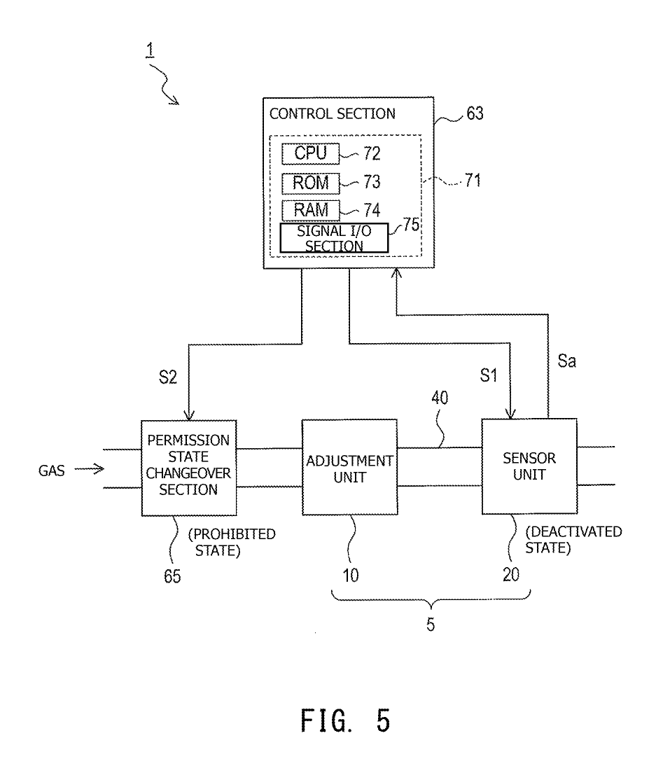

Also, in the case the control section 63 determines, during execution of the detection state setting process, that the present period is not the NO detection period, for setting the state of gas detection by the sensor unit 20 to the detection suspended state, the control section 63 outputs the first command signal S1 so as to set the state of the sensor unit 20 to the deactivated state and outputs the second command signal S2 so as to set the gas supply state of the permission state changeover section 65 to the prohibited state (the gas supply changeover process). As a result, as shown in FIG. 5, the gas detection apparatus 1 can set the state of the permission state changeover section 65 to a state in which the gas cannot pass through the permission state changeover section 65, and can stop the NO₂ detection at the sensor unit 20 by establishing a state in which the conversion of the gas at the adjustment unit 10 is stopped, whereby the supply of the converted gas G2 to the sensor unit 20 is stopped.

### 1-4. Effects

As described above, in the gas detection apparatus 1 of the present embodiment, the control section 63 is configured such that, in the case where the control section 63 determines, during execution of the detection state setting process, that the present period is the NO detection period, the control section 63 sets the state of gas detection by the sensor unit 20 to the detection executed state. Also, in the case where the control section 63 determines, during execution of the detection state setting process, that the present period is not the NO detection period, the control section 63 sets the state of gas detection by the sensor unit 20 to the detection suspended state.

As described above, the state of gas detection by the sensor unit 20 is set to the detection suspended state during periods which are not the NO detection period. Therefore, the reaction between the converted gas G2 and the sensor unit 20 does not occur in a period between the startup of the gas detection apparatus 1 and the beginning of the detection period (in other words, a period which is not the detection period). As a result, a decrease in accuracy in detecting NO₂ at the sensor unit 20 can be suppressed.

Accordingly, the gas detection apparatus 1 can suppress a decrease in accuracy in detecting NO₂ at the sensor unit 20, and thus can suppress a decrease in accuracy in detecting NO.

The gas detection apparatus 1 includes the permission state changeover section 65. The permission state changeover section 65 is configured to switch the state of gas supply to the gas sensor 5 (the adjustment unit 10 and the sensor unit 20) to either of the permission state in which the converted gas G2 is supplied to the sensor unit 20 and the prohibited state in which the converted gas G2 is not supplied to the sensor unit 20.

The control section 63 is configured to operate as follows. In the case where the control section 63 determines, during execution of the detection state setting process, that the present period is the NO detection period, for setting the state of gas detection by the sensor unit 20 to the detection executed state, the control section 63 sets at least the gas supply state of the permission state changeover section 65 to the permission state. Also, in the case the control section 63 determines, during execution of the detection state setting process, that the present period is not the NO detection period, for setting the state of gas detection by the sensor unit 20 to the detection suspended state, the control section 63 sets at least the gas supply state of the permission state changeover section 65 to the prohibited state.

As described above, as a method of controlling the state of gas detection by the sensor unit 20 to the detection executed state or the detection suspended state, the control section 63 can employ a method of switching the state of gas supply to the gas sensor 5 (the adjustment unit 10 and the sensor unit 20) to the permission state or the prohibited state by controlling the permission state changeover section 65.

As a result, the time during which NO₂ is supplied to the sensor unit 20 can be shortened by switching the state of supply of the converted gas G2 to the sensor unit 20 by controlling the permission state changeover section 65, without switching the state of the sensor unit 20 (between the activated state and the deactivated state).

Therefore, even in the case where NO detection is performed over a long period of time, the gas detection apparatus 1 can suppress a decrease in accuracy in detecting NO₂ at the sensor unit 20. This is because the gas detection apparatus 1 can prevent the sensor unit 20 from needlessly being exposed to the converted gas G2 in periods which are not the NO detection period.

Notably, in the gas detection apparatus 1, the prohibited state in which the converted gas G2 is not supplied to the sensor unit 20 is a state in which the supply of gas to the adjustment unit 10 is stopped (prohibited) and the supply of the converted gas G2 to the sensor unit 20 is stopped (prohibited).

Such a prohibited state is readily realized by stopping the supply of gas to the adjustment unit 10 without changing the gas flow channel extending from the adjustment unit 10 to the sensor unit 20.

Also, the gas detection apparatus 1 includes the first heater 24b. The first heater 24b is configured to switch the temperature of the sensor unit 20 (specifically, the detection section 24a) between the activation temperature (a temperature at which the sensor unit 20 can detect NO₂) and the deactivation temperature (a temperature at which the sensor unit 20 cannot detect NO₂) by adjusting the amount of generated heat based on the first command signal S1 (supply power amount) from the control section 63.

In the case where the control section 63 determines, during execution of the detection state setting process, that the present period is the NO detection period, for setting the state of gas detection by the sensor unit 20 to the detection executed state, the control section 63 controls at least the first heater 24b such that the temperature of the sensor unit 20 becomes the activation temperature. Also, in the case where the control section 63 determines, during execution of the detection state setting process, that the present period is not the NO detection period, for setting the state of gas detection by the sensor unit 20 to the detection suspended state, the control section 63 controls at least the first heater 24b such that the temperature of the sensor unit 20 becomes the deactivation temperature.

As described above, as a method of controlling the state of gas detection by the sensor unit 20 to the detection executed state or the detection suspended state, the gas detection apparatus 1 of the present embodiment can employ a method of switching the temperature of the sensor unit 20 (specifically, the detection section 24a) to the activation temperature or the deactivation temperature by controlling the first heater 24b in addition to the method of switching the state of supply of the converted gas G2 to the sensor unit 20 by controlling the permission state changeover section 65.

As a result, even in the case where NO detection is performed over a long period of time, the gas detection apparatus 1 can suppress a decrease in accuracy in detecting NO₂ at the sensor unit 20. This is because in periods which are not the NO detection period, the period of time during which the sensor unit 20 is in the activated state can be shortened and the sensor unit 20 is prevented from needlessly being exposed to the converted gas G2. Also, the amount of electric power consumed, without purpose, by the first heater 24b can be reduced by switching the temperature of the sensor unit 20 (specifically, the detection section 24a) from the activation temperature to the deactivation temperature.

### 1-5. Corresponding Terms

Terms used in describing the embodiment and corresponding features of the invention will be described as follows.

The gas detection apparatus 1 corresponds to the gas detection apparatus of the invention; exhaled air corresponds to the gas under measurement of the invention; NO corresponds to the first gas component of the invention; and NO₂ corresponds to the second gas component of the invention. The adjustment unit 10 corresponds to the gas conversion section of the invention; the sensor unit 20 corresponds to the gas detection section of the invention; the control section 63 corresponds to the detection state setting section of the invention; the permission state changeover section 65 corresponds to the permission state changeover section of the invention; and the first heater 24b corresponds to the reaction state changeover section of the invention.

### 2. Second Embodiment

### 2-1. Overall Configuration

A second gas detection apparatus 101 which includes a moving direction changeover section 66 in place of the permission state changeover section 65 in the gas detection apparatus 1 of the first embodiment will be described as a second embodiment.

Notably, of the constituent elements of the second gas detection apparatus 101 of the second embodiment, constituent elements identical with those of the gas detection apparatus 1 of the first embodiment will be described using the same reference numerals. In the following description, a portion of the second embodiment different from the first embodiment will mainly be described.

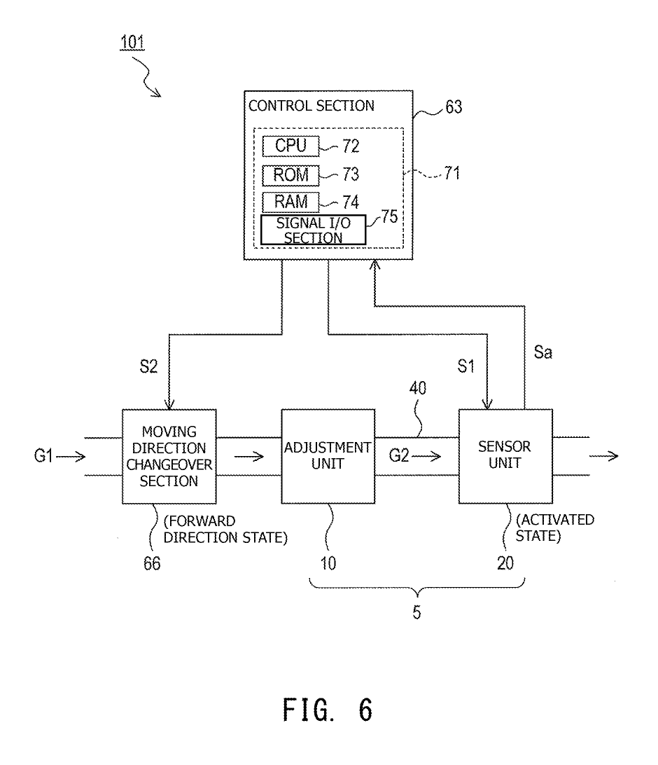

As shown in FIG. 6, the second gas detection apparatus 101 includes the gas sensor 5 for measuring NOx contained in the gas under measurement G1 the control section 63 for controlling the gas sensor 5, and the moving direction changeover section 66 for switching the moving direction of gas supplied to the gas sensor 5.

The gas sensor 5 includes the adjustment unit 10 and the sensor unit 20.

The adjustment unit 10 includes a catalyst (MCR) for converting NO contained in the gas under measurement G1 supplied from the moving direction changeover section 66 to NO₂.

The control section 63 is configured to control the state of gas detection by the sensor unit 20 and receive a detection signal Sa which changes with the NO₂ concentration detected by the sensor unit 20.

The control section 63 is configured to control at least either of the state of the sensor unit 20 (between an activated state and an deactivated state) and the state of the moving direction changeover section 66 when the control section 63 controls the state of gas detection by the sensor unit 20.

The control section 63 can set the moving direction of the gas supplied from the moving direction changeover section 66 to the gas sensor 5 to either of the forward direction (i.e., set the moving direction changeover section 66 to a forward direction state) and the reverse direction (i.e., set the moving direction changeover section 66 to a reverse direction state) by controlling the gas moving direction of the moving direction changeover section 66 through output of the second command signal S2.

The moving direction changeover section 66 is configured to supply the gas under measurement G1 to the gas sensor 5 (specifically, the adjustment unit 10) when the moving direction changeover section 66 is set to the forward direction state. More specifically, when a timing for supplying exhaled air to the gas sensor 5 as the gas under measurement G1 has come after the startup of the second gas detection apparatus 101, the moving direction changeover section 66 supplies the gas under measurement G1 to the gas sensor 5 over a period of time during which the detection by the gas sensor 5 is performed. The moving direction changeover section 66 includes, for example, a blower whose blowing direction can be switched. Thus, the moving direction changeover section 66 can switch the moving direction of the gas supplied to the gas sensor 5 to either of the forward direction and the reverse direction.

The forward direction state of the moving direction changeover section 66 is a state in which, as shown in FIG. 6, the gas under measurement G1 is supplied from the moving direction changeover section 66 to the gas sensor 5 (the adjustment unit 10) and is also a state in which the converted gas G2 is supplied from the adjustment unit 10 to the sensor unit 20.

The moving direction changeover section 66 is configured to draw the gas from the gas sensor 5 (specifically, the adjustment unit 10) when the moving direction changeover section 66 is set to the reverse direction state.

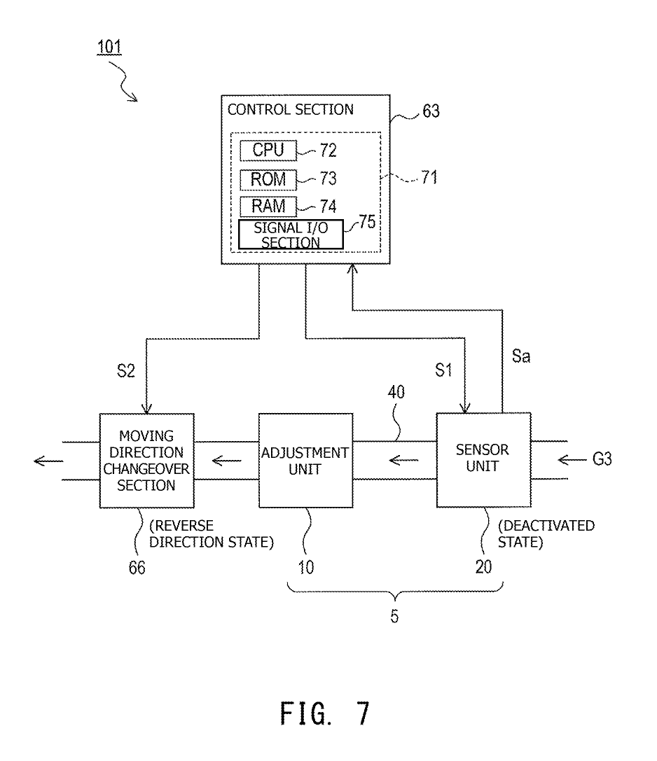

The reverse direction state of the moving direction changeover section 66 is a state in which, as shown in FIG. 7, the moving direction changeover section 66 draws the gas inside the adjustment unit 10 and is also a state in which, due to negative pressure, the gas inside the sensor unit 20 is drawn to the adjustment unit 10 and the atmosphere G3 is drawn into the sensor unit 20. In other words, the reverse direction state of the moving direction changeover section 66 is a state in which the gas under measurement G1 is not supplied to the gas sensor 5 (the adjustment unit 10) and is also a state in which the converted gas G2 is not supplied from the adjustment unit 10 to the sensor unit 20.

### 2-2. Control Section

As one of the various processes, the control section 63 executes a process of setting the gas moving direction of the moving direction changeover section 66 to either of the forward direction and the reverse direction by outputting the second command signal S2 (hereinafter this process will also be referred to as a “gas moving direction changeover process”).

Further, as one of the various processes, the control section 63 executes a process of setting the state of gas detection by the sensor unit 20 to either of the detection executed state and the detection suspended state depending on whether or not the present period is the NO detection period (hereinafter this process will also be referred to as a “detection state setting process”).

In the case where the control section 63 determines, during execution of the detection state setting process, that the present period is the NO detection period, for setting the state of gas detection by the sensor unit 20 to the detection executed state, the control section 63 outputs the first command signal S1 so as to set the state of the sensor unit 20 to the activated state (the sensor state setting process) and outputs the second command signal S2 so as to set the gas moving direction of the moving direction changeover section 66 to the forward direction (the gas supply changeover process). As a result, as shown in FIG. 6, the second gas detection apparatus 101 can be set to a state in which the moving direction changeover section 66 can supply the gas under measurement G1 to the gas sensor 5 (the adjustment unit 10), whereby it becomes possible to supply to the sensor unit 20 the converted gas G2 converted from the gas under measurement G1 at the adjustment unit 10. Therefore, the second gas detection apparatus 101 can detect NO₂ of the converted gas G2 at the sensor unit 20.

Also, in the case the control section 63 determines, during execution of the detection state setting process, that the present period is not the NO detection period, for setting the state of gas detection by the sensor unit 20 to the detection suspended state, the control section 63 outputs the first command signal S1 so as to set the state of the sensor unit 20 to the deactivated state (the sensor state setting process) and outputs the second command signal S2 so as to set the gas moving direction of the moving direction changeover section 66 to the reverse direction (the gas supply changeover process). As a result, as shown in FIG. 7, the second gas detection apparatus 101 is set to a state in which, due to negative pressure, the atmosphere G3 is drawn into the sensor unit 20 and is then drawn into the adjustment unit 10, and the gas having passed through the adjustment unit 10 is drawn into the moving direction changeover section 66. In this manner, the second gas detection apparatus 101 can stop the NO₂ detection at the sensor unit 20 as a result of establishing a state in which conversion of the gas under measurement G1 at the adjustment unit 10 is stopped, whereby the supply of the converted gas G2 to the sensor unit 20 is stopped. As a result, during periods which are not the NO detection period, the converted gas G2 from which miscellaneous gases have been removed is not supplied to the sensor unit 20.

### 2-3. Effects

As described above, in the second gas detection apparatus 101, the control section 63 is configured such that, in the case where the control section 63 determines, during execution of the detection state setting process, that the present period is the NO detection period, the control section 63 sets the state of gas detection by the sensor unit 20 to the detection executed state. Also, in the case where the control section 63 determines, during execution of the detection state setting process, that the present period is not the NO detection period, the control section 63 sets the state of gas detection by the sensor unit 20 to the detection suspended state.

Also, the second gas detection apparatus 101 includes the moving direction changeover section 66. The moving direction changeover section 66 is configured to switch the moving direction of the gas supplied to the gas sensor 5 (the adjustment unit 10 and the sensor unit 20) to the forward direction so as to supply the converted gas G2 to the sensor unit 20 or to the reverse direction so as to prevent the supply of converted gas G2 to the sensor unit 20.

The control section 63 is configured to operate as follows. In the case where the control section 63 determines, during execution of the detection state setting process, that the present period is the NO detection period, for setting the state of gas detection by the sensor unit 20 to the detection executed state, the control section 63 sets at least the gas moving direction of the moving direction changeover section 66 to the forward direction. Also, in the case the control section 63 determines, during execution of the detection state setting process, that the present period is not the NO detection period, for setting the state of gas detection by the sensor unit 20 to the detection suspended state, the control section 63 sets at least the gas moving direction of the moving direction changeover section 66 to the reverse direction.

As described above, as a method of controlling the state of gas detection by the sensor unit 20 to the detection executed state or the detection suspended state, the control section 63 can employ a method of switching the moving direction of the gas supplied to the gas sensor 5 (the adjustment unit 10 and the sensor unit 20) to the forward direction or the reverse direction by controlling the moving direction changeover section 66.

As a result, the time during which NO₂ is supplied to the sensor unit 20 can be shortened by switching the state of supply of the converted gas G2 to the sensor unit 20 by controlling the moving direction changeover section 66, without switching the state of the sensor unit 20 (between the activated state and the deactivated state).

Therefore, even in the case where NO detection is performed over a long period of time, the second gas detection apparatus 101 can suppress a decrease in accuracy in detecting NO₂ at the sensor unit 20. This is because the second gas detection apparatus 101 can prevent the sensor unit 20 from needlessly being exposed to the converted gas G2 in periods which are not the NO detection period.

Notably, in the second gas detection apparatus 101, the supply suspended state in which the converted gas G2 is not supplied to the sensor unit 20 is a state in which the atmosphere G3 (gas not to be detected) which is not the converted gas G2 is supplied to the sensor unit 20 and the atmosphere G3 having passed through the sensor unit 20 is supplied to the adjustment unit 10.

Such a supply suspended state is readily realized by changing the gas moving direction in the gas flow channel between the adjustment unit 10 and the sensor unit 20 from the forward direction (the gas moving direction in the supply executed state) to the opposite direction (the gas moving direction in the supply suspended state).

### 2-4. Corresponding Terms

Terms used in describing the present embodiment and corresponding features of the invention will be described as follows.

The second gas detection apparatus 101 corresponds to the gas detection apparatus of the invention; the atmosphere G3 corresponds to the gas not to be detected of the invention; and the moving direction changeover section 66 corresponds to the supply state changeover section of the invention.

### 3. Third Embodiment

### 3-1. Overall Configuration

A third gas detection apparatus 201 including a flow channel changeover section 85 which switches a supply source flow channel for the gas supplied to the sensor unit 20 will be described as a third embodiment.

Notably, of the constituent elements of the third gas detection apparatus 201 of the third embodiment, constituent elements identical with those of the gas detection apparatus 1 of the first embodiment will be described using the same reference numerals. In the following description, a portion of the third embodiment different from the first embodiment will be mainly described.

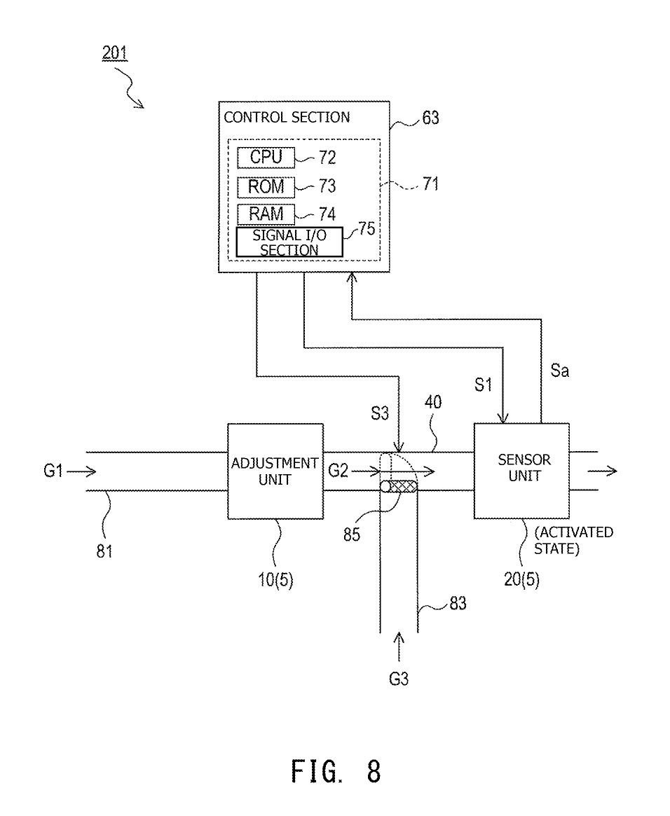

As shown in FIG. 8, the third gas detection apparatus 201 includes the gas sensor 5 for measuring NOx contained in the gas under measurement G1, the control section 63 for controlling the gas sensor 5, and the flow channel changeover section 85 which switches the supply source flow channel for the gas supplied to the sensor unit 20 of the gas sensor 5.

The gas sensor 5 includes the adjustment unit 10 and the sensor unit 20.

The adjustment unit 10 includes a catalyst (MCR) for converting NO contained in the gas under measurement G1 supplied from a first gas introduction port 81 to NO₂.

The flow channel changeover section 85 is provided in a gas flow pipe 40 which connects the adjustment unit 10 and the sensor unit 20. The flow channel changeover section 85 is configured to switch the supply source flow channel for the gas supplied to the sensor unit 20 to either of a flow channel communicating with the adjustment unit 10 and a flow channel communicating with a second gas introduction port 83. Namely, the flow channel changeover section 85 is configured to switch its state to either of a detection-time flow channel state in which the gas supply source flow channel is set to the flow channel communicating with the adjustment unit 10 and a suspended-time flow channel state in which the gas supply source flow channel is set to the flow channel communicating with the second gas introduction port 83.

The control section 63 is configured to control the state of gas detection by the sensor unit 20 and receive a detection signal Sa which changes with the NO₂ concentration detected by the sensor unit 20.

The control section 63 is configured to control at least either of the state of the sensor unit 20 (between an activated state and an deactivated state) and the state of the flow channel changeover section 85 when the control section 63 controls the state of gas detection by the sensor unit 20.

The control section 63 can switch the gas supplied to the sensor unit 20 to either of the converted gas G2 and the atmosphere G3 by controlling the flow channel set state of the flow channel changeover section 85 to either of the detection-time flow channel state and the suspended-time flow channel state through output of a third command signal S3.

The flow channel changeover section 85 is configured to supply the converted gas G2 to the sensor unit 20 when the flow channel changeover section 85 is set to the detection-time flow channel state. The flow channel changeover section 85 includes, for example, a three way valve or the like. Thus, the flow channel changeover section 85 can switch its flow channel set state to either of the detection-time flow channel state and the suspended-time flow channel state.

The detection-time flow channel state of the flow channel changeover section 85 is a state in which, as shown in FIG. 8, the gas under measurement G1 introduced from the first gas introduction port 81 is supplied to the gas sensor 5 (the adjustment unit 10), and is also a state in which the converted gas G2 is supplied from the adjustment unit 10 to the sensor unit 20.

The flow channel changeover section 85 is configured to supply the atmosphere G3 to the sensor unit 20 when the flow channel changeover section 85 is set to the suspended-time flow channel state.

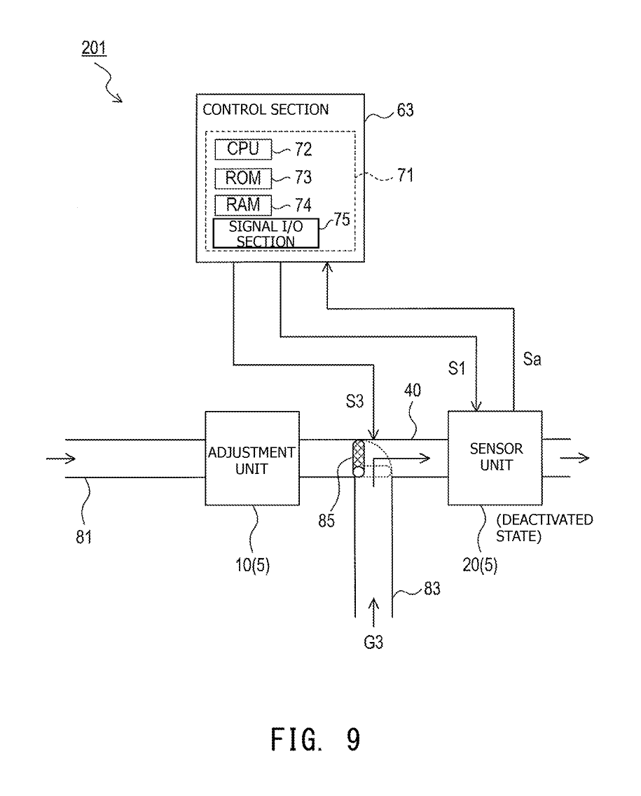

The suspended-time flow channel state of the flow channel changeover section 85 is a state in which, as shown in FIG. 9, the gas introduced from the first gas introduction port 81 (specifically, the converted gas G2 produced as a result of conversion at the adjustment unit 10) is stopped by the flow channel changeover section 85, and the atmosphere G3 introduced from the second gas introduction port 83 is supplied to the sensor unit 20.

### 3-2. Control Section

As one of the various processes, the control section 63 executes a process of switching the flow channel set state of the flow channel changeover section 85 to either of the detection-time flow channel state and the suspended-time flow channel state by outputting the third command signal S3 (hereinafter this process will also be referred to as a “gas supply changeover process”).

Further, as one of the various processes, the control section 63 executes a process of setting the state of gas detection by the sensor unit 20 to either of the detection executed state and the detection suspended state depending on whether or not the present period is the NO detection period (hereinafter this process will also be referred to as a “detection state setting process”).

In the case where the control section 63 determines, during execution of the detection state setting process, that the present period is the NO detection period, for setting the state of gas detection by the sensor unit 20 to the detection executed state, the control section 63 outputs the first command signal S1 so as to set the state of the sensor unit 20 to the activated state (the sensor state setting process) and outputs the third command signal S3 so as to set the flow channel set state of the flow channel changeover section 85 to the detection-time flow channel state (the gas supply changeover process). As a result, as shown in FIG. 8, it becomes possible for the third gas detection apparatus 201 to supply the gas under measurement G1 to the adjustment unit 10 of the gas sensor 5 and supply the converted gas G2 (converted from the gas under measurement G1 at the adjustment unit 10) to the sensor unit 20. Therefore, the third gas detection apparatus 201 can detect NO₂ of the converted gas G2 at the sensor unit 20.

Also, in the case the control section 63 determines, during execution of the detection state setting process, that the present period is not the NO detection period, for setting the state of gas detection by the sensor unit 20 to the detection suspended state, the control section 63 outputs the first command signal S1 so as to set the state of the sensor unit 20 to the deactivated state (the sensor state setting process) and outputs the third command signal S3 so as to set the flow channel set state of the flow channel changeover section 85 to the suspended-time flow channel state (the gas supply changeover process). As a result, as shown in FIG. 9, the third gas detection apparatus 201 is set to a state in which the atmosphere G3 introduced from the second gas introduction port 83 is supplied to the sensor unit 20. In this manner, a state in which converted gas G2 is not supplied to the sensor unit 20 is established, whereby the NO₂ detection at the sensor unit 20 can be stopped. As a result, during periods which are not the NO detection period, converted gas G2 from which miscellaneous gases have been removed is not supplied to the sensor unit 20.

### 3-3. Effects

As described above, in the third gas detection apparatus 201, the control section 63 is configured such that, in the case where the control section 63 determines, during execution of the detection state setting process, that the present period is the NO detection period, the control section 63 sets the state of gas detection by the sensor unit 20 to the detection executed state. Also, in the case where the control section 63 determines, during execution of the detection state setting process, that the present period is not the NO detection period, the control section 63 sets the state of gas detection by the sensor unit 20 to the detection suspended state.

Also, the third gas detection apparatus 201 includes the flow channel changeover section 85. The flow channel changeover section 85 is configured to switch the state of itself to either of the detection-time flow channel state in which the gas supply source flow channel is set to the flow channel communicating with the adjustment unit 10 and the suspended-time flow channel state in which the gas supply source flow channel is set to the flow channel communicating with the second gas introduction port 83.

The control section 63 is configured to operate as follows. In the case where the control section 63 determines, during execution of the detection state setting process, that the present period is the NO detection period, for setting the state of gas detection by the sensor unit 20 to the detection executed state, the control section 63 sets at least the flow channel set state of the flow channel changeover section 85 to the detection-time flow channel state. Also, in the case the control section 63 determines, during execution of the detection state setting process, that the present period is not the NO detection period, for setting the state of gas detection by the sensor unit 20 to the detection suspended state, the control section 63 sets at least the flow channel set state of the flow channel changeover section 85 to the suspended-time flow channel state.

As described above, as a method of controlling the state of gas detection by the sensor unit 20 to the detection executed state or the detection suspended state, the control section 63 can employ a method of switching the flow channel set state to the detection-time flow channel state or the suspended-time flow channel state by controlling the flow channel changeover section 85.

As a result, the time during which NO₂ is supplied to the sensor unit 20 can be shortened by switching the state of supply of the converted gas G2 to the sensor unit 20 by controlling the flow channel changeover section 85, without switching the state of the sensor unit 20 (between the activated state and the deactivated state).

Therefore, even in the case where NO detection is performed over a long period of time, the third gas detection apparatus 201 can suppress a decrease in accuracy in detecting NO₂ at the sensor unit 20. This is because the third gas detection apparatus 201 can prevent the sensor unit 20 from needlessly being exposed to the converted gas G2 in periods which are not the NO detection period.

Notably, in the third gas detection apparatus 201, the supply suspended state in which the converted gas G2 is not supplied to the sensor unit 20 is a state in which the supply of the converted gas G2 to the sensor unit 20 is stopped and the atmosphere G3 which is not the converted gas G2 is supplied to the sensor unit 20.

Such a supply suspended state is readily realized by stopping the supply of converted gas G2 from the adjustment unit 10 to the sensor unit 20, and supplying the atmosphere G3 to the gas flow channel which connects the adjustment unit 10 and the sensor unit 20.

### 3-4. Corresponding Terms

Terms used in describing the present embodiment and corresponding features of the invention will be described as follows.

The third gas detection apparatus 201 corresponds to the gas detection apparatus of the invention; the atmosphere G3 corresponds to the gas not to be detected of the invention; and the flow channel changeover section 85 corresponds to the supply state changeover section of the invention.

### 4. Other Embodiments

Certain embodiments of the present invention have been described; however, the present invention is not limited thereto and may be implemented in various forms without departing from the scope of the present invention.

For example, in the above-described first embodiment, the gas detection apparatus 1 is configured such that, when the gas detection apparatus 1 determines that the present period is not the NO detection period and sets the state of gas detection by the sensor unit 20 to the detection suspended state, the gas detection apparatus 1 executes two processes (the process of setting the state of the sensor unit 20 to the deactivated state and the process of setting the gas supply state of the permission state changeover section 65 to the prohibited state). However, the gas detection apparatus of the present invention is not limited to the gas detection apparatus 1 configured as described above. For example, the gas detection apparatus 1 may be configured such that, when the gas detection apparatus 1 sets the state of gas detection by the sensor unit 20 to the detection suspended state, the gas detection apparatus 1 executes one process (only the process of setting the state of the sensor unit 20 to the deactivated state, or only the process of setting the gas supply state of the permission state changeover section 65 to the prohibited state).

In the case where the gas detection apparatus 1 is configured to execute a single process so as to set the state of gas detection by the sensor unit 20 to the detection suspended state, the configuration of the apparatus can be simplified as compared with the case where the gas detection apparatus 1 is configured to execute two processes. Also, in the case where the state of the gas sensor 20 is switched to either of the activated state and the deactivated state, the time required for the state switching may become long. In contrast, the switching of the state of the permission state changeover section 65 can be performed within a short period of time. Therefore, by employing the configuration of executing only the process of switching the state of the permission state changeover section 65, it is possible to yield the advantage that the process of switching the state of gas detection by the sensor unit 20 to either of the detection executed state and the detection suspended state can be executed within a short period of time.

Similarly, in the above-described second embodiment as well, the configuration of the gas detection apparatus is not limited to the configuration in which the apparatus executes two processes so as to set the state of gas detection by the sensor unit 20 to the detection suspended state. Rather, the gas detection apparatus may be configured to execute a single processes (only the process of setting the state of the sensor unit 20 to the deactivated state, or only the process of setting the gas moving direction of the moving direction changeover section 66 to the reverse direction). In the above-described third embodiment as well, the configuration of the gas detection apparatus is not limited to the configuration in which the apparatus executes two processes so as to set the state of gas detection by the sensor unit 20 to the detection suspended state. Rather, the gas detection apparatus may be configured to execute a single processes (only the process of setting the state of the sensor unit 20 to the deactivated state, or only the process of setting the flow channel set state of the flow channel changeover section 85 to the suspended-time flow channel state).

In the above-described embodiments, the state of gas detection by the sensor unit 20 is controlled to the detection executed state or the detection suspended state by controlling the pen fission state changeover section 65 or controlling the first heater 24b (the temperature of the detection section 24a). The gas detection apparatus of the present invention is not limited to the embodiments, and may be configured to control the state of gas detection by the sensor unit 20 to the detection executed state or the detection suspended state by switching the gas conversion state at the adjustment unit 10 to the conversion possible state or the no conversion state by controlling the second heater 14c.

Specifically, the control section 63 may output a fourth command signal S4 to the adjustment unit 10 so as to control the gas conversion state at the adjustment unit 10 to thereby switch the gas supplied to the sensor unit 20 between the gas under measurement G1 (without conversion to the converted gas G2) and the converted gas G2 converted from the gas under measurement G1. By switching the gas supplied to the sensor unit 20 in this manner, it is possible to control the state of gas detection by the sensor unit 20 to the detection executed state or the detection suspended state.

Namely, during the period of detection by the gas sensor 5, by setting the first catalyst 14a and the second catalyst 14b to a catalyst reaction temperature by controlling the heat generation amount of the second heater 14c, the adjustment unit 10 can be controlled to the conversion possible state, whereby the converted gas G2 can be supplied to the sensor unit 20. As a result, the state of gas detection by the sensor unit 20 can be controlled to the detection executed state. Also, during periods which are not the period of detection by the gas sensor 5, by setting the first catalyst 14a and the second catalyst 14b to a catalyst non-reaction temperature (temperature at which the catalysts cannot exhibit the catalytic function) by controlling the heat generation amount of the second heater 14c, the adjustment unit 10 can be controlled to the no conversion state, whereby the gas (e.g., the atmosphere) introduced to the adjustment unit 10 can be supplied to the sensor unit 20 as is. As a result, the state of gas detection by the sensor unit 20 can be controlled to the detection suspended state. Therefore, during periods which are not the NO detection period, the converted gas G2 from which miscellaneous gases have been removed is not supplied to the sensor unit 20. In this case, the control section 63 corresponds to the supply state changeover section; and the second heater 14c corresponds to the conversion state changeover section.

The function of a single constituent element in each embodiment may be realized by a plurality of constituent elements, or the functions of a plurality of constituent elements may be realized by a single constituent element. A portion of the configuration of each embodiment may be omitted. At least a portion of the configuration of each embodiment may be used in other embodiments in addition to or in place of the constituent element(s) thereof. Notably, all embodiments which fall within the technical idea determined from the wording in the claims are the embodiments of the present invention.

The present invention can be realized in various forms, such as the above-described microcomputer, a system which includes the microcomputer as a constituent element, a program for causing a computer to function as the microcomputer, a non-transitory substantial recording medium, such as semiconductor memory, on which the program is recorded, and a concentration calculation method.

The invention has been described in detail with reference to the above embodiments. However, the invention should not be construed as being limited thereto. It should further be apparent to those skilled in the art that various changes in form and detail of the invention as shown and described above may be made. It is intended that such changes be included within the spirit and scope of the claims appended hereto.

What is claimed is:

1. A gas detection apparatus for detecting the concentration of a first gas component contained in a gas under measurement, comprising:
a gas conversion section which converts at least a portion of the first gas component contained in the gas under measurement to a second gas component such that the ratio between partial pressures of the first gas component and the second gas component coincides with that in an equilibrium state;
a gas detection section to which a converted gas produced as a result of conversion at the gas conversion section is supplied and whose electrical characteristics change with a change in the concentration of the second gas component in the converted gas when the gas detection section is brought into an activated state in which the gas detection section can detect the second gas component; and
a detection state setting section which sets the state of gas detection by the gas detection section,
wherein during a detection period in which the first gas component is detected, the detection state setting section sets the state of gas detection by the gas detection section to a detection executed state in which the gas detection section can detect the second gas component of the converted gas, and during periods which are not the detection period, the detection state setting section sets the state of gas detection by the gas detection section to a detection suspended state in which the gas detection section cannot detect the second gas component of the converted gas.

2. The gas detection apparatus as claimed in claim 1, further comprising a supply state changeover section which switches the state of gas supply to the gas detection section to either of a supply executed state in which the converted gas is supplied to the gas detection section and a supply suspended state in which the converted gas is not supplied to the gas detection section, and a gas not to be detected which is not the converted gas is supplied to the gas detection section,
wherein during the detection period, the detection state setting section controls the supply state changeover section such that the state of gas supply to the gas detection section is set to the supply executed state, and during periods which are not the detection period, the detection state setting section controls the supply state changeover section such that the state of gas supply to the gas detection section is set to the supply suspended state.

3. The gas detection apparatus as claimed in claim 2, wherein the supply suspended state is a state in which the gas not to be detected is supplied to the gas detection section from a downstream side of the gas detection section and the gas not to be detected which has passed through the gas detection section is supplied to the gas conversion section.

4. The gas detection apparatus as claimed in claim 2, wherein the supply suspended state is a state in which the supply of the converted gas to the gas detection section is stopped and the gas not to be detected is supplied to a passage between the gas conversion section and the gas detection section.

5. The gas detection apparatus as claimed in claim 2, further comprising a conversion state changeover section which switches the state of the gas conversion section to either of a conversion possible state in which the gas supplied to the gas conversion section can be converted to the converted gas and a no conversion state in which the gas supplied to the gas conversion section passes through the gas conversion section without being converted to the converted gas,
wherein during the detection period, the supply state changeover section controls the conversion state changeover section such that the state of the gas conversion section becomes the conversion possible state, and during periods which are not the detection period, the supply state changeover section controls the conversion state changeover section such that the state of the gas conversion section becomes the no conversion state.

6. The gas detection apparatus as claimed in claim 1, further comprising a reaction state changeover section which switches the state of the gas detection section to either of a reaction executed state in which the gas detection section reacts with the second gas component and a reaction suspended state in which the gas detection section does not react with the second gas component,
the reaction state changeover section being configured to set the state of the gas detection section to the reaction executed state by controlling the temperature of the gas detection section to an activation temperature at which the gas detection section can detect the second gas component and set the state of the gas detection section to the reaction suspended state by controlling the temperature of the gas detection section to a deactivation temperature at which the gas detection section does not detect the second gas component,
wherein during the detection period, the detection state setting section controls the reaction state changeover section such that the state of the gas detection section becomes the reaction executed state, and during periods which are not the detection period, the detection state setting section controls the reaction state changeover section such that the state of the gas detection section becomes the reaction suspended state.

7. The gas detection apparatus as claimed in claim 1, further comprising a permission state changeover section which switches the state of gas supply to the gas conversion section between a permission state in which the gas supply is permitted and a prohibition state in which the gas supply is prohibited,
wherein the detection state setting section controls the permission state changeover section such that the state of gas supply to the gas conversion section is switched to the permission state during the detection period and is switched to the prohibition state during periods which are not the detection period.

8. The gas detection apparatus as claimed in claim 1, wherein
the gas conversion section includes a catalyst for replacing NO in the gas under measurement with NO₂ and is configured to convert NO which is the first gas component to NO₂ which is the second gas component, and
the gas detection section is configured such that its electrical characteristics change with a change in the concentration of NO₂ which is the second gas component.
